SEQUENCE LISTING (20 sequences)

SEQ ID NO: 1 - protein, 120 aa, Artificial Sequence
  VH domain of a CEA-specific scFv used in the examples
QVKLEQSGAEVVKPGASVKLSCKASGFNIKDSYMHWLRQGPGQCLEWIGWIDPENGDTEYAPKFQGKATFTTDTSANTAYLGLSSLRPEDTAVYYCNEGTPTGPYYFDYWGQGTLVTVSS

SEQ ID NO: 2 - protein, 106 aa, Artificial Sequence
  VL domain of a CEA-specific scFv used in the examples
ENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGCGTKLEIK

SEQ ID NO: 3 - protein, 483 aa, Artificial Sequence
  CEA-specific scFv-Fc used in the examples
MAQVKLEQSGAEVVKPGASVKLSCKASGFNIKDSYMHWLRQGPGQCLEWIGWIDPENGDTEYAPKFQGKATFTTDTSANTAYLGLSSLRPEDTAVYYCNEGTPTGPYYFDYWGQGTLVTVSSGGGGSGGGGSGGGGSGGGGSENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGCGTKLEIKRSSRAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 113 aa, Artificial Sequence
  VH domain of a GD2-specific scFv (clone 14G2A) used in the examples
EVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 5 - protein, 113 aa, Artificial Sequence
  VL domain of a GD2-specific scFv (clone 14G2A) used in the examples
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 6 - protein, 474 aa, Artificial Sequence
  GD2-specific scFv-Fc (clone 14G2A) used in the examples
EVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRSAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 248 aa, Artificial Sequence
  CEA-specific scFv used in the examples
MAQVKLEQSGAEVVKPGASVKLSCKASGFNIKDSYMHWLRQGPGQCLEWIGWIDPENGDTEYAPKFQGKATFTTDTSANTAYLGLSSLRPEDTAVYYCNEGTPTGPYYFDYWGQGTLVTVSSGGGGSGGGGSGGGGSGGGGSENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGCGTKLEIK

SEQ ID NO: 8 - protein, 241 aa, Artificial Sequence
  GD2-specific scFv (clone 14G2A) used in the examples
EVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGGGGSGGGGSGGGGSDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 9 - protein, 122 aa, Artificial Sequence
  VH domain of a B7H3-specific scFv
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 10 - protein, 107 aa, Artificial Sequence
  VL domain of a B7H3-specific scFv
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 11 - protein, 477 aa, Artificial Sequence
  B7H3-specific scFv-Fc
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRSAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 244 aa, Artificial Sequence
  B7H3-specific scFv
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 13 - protein, 121 aa, Artificial Sequence [sequence omitted]

SEQ ID NO: 14 - protein, 106 aa, Artificial Sequence [sequence omitted]

SEQ ID NO: 15 - protein, 475 aa, Artificial Sequence
  CD20-specific scFv-Fc
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRSAAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 242 aa, Artificial Sequence
  CD20-specific scFv
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 17 - protein, 744 aa, Artificial Sequence
  GD2 specific IgG1 (clone 14G2A) used in the examples, having a cleavage site between the light and heavy chains
MLPSQLIGFLLLWVPASRGDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRAKRGKPIPNPLLGLDSTSGSGSGATNFSLLKQAGDVEENPGPMDWTWRILFLVAAATGAHSEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 239 aa, Artificial Sequence
  light chain of the GD2 IgG1 of SEQ ID NO 17
MLPSQLIGFLLLWVPASRGDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 43 aa, Artificial Sequence
  cleavage sequence (Furin-V5-SG-P2A) of the GD2 IgG1 of SEQ ID NO 17
RAKRGKPIPNPLLGLDSTSGSGSGATNFSLLKQAGDVEENPGP

SEQ ID NO: 20 - protein, 462 aa, Artificial Sequence
  heavy chain of the GD2 IgG1 of SEQ ID NO 17
MDWTWRILFLVAAATGAHSEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2023/0049025 A1
(43) Pub. Date: Feb. 16, 2023

(54) ENGINEERED IMMUNE CELLS

(71) Applicant: UCL Business Ltd, Loncon (GB)

(72) Inventors: Jonathan Fisher, London (GB); Kerry Chester, London (GB); John Anderson, London (GB); Enrique Miranda Rota, London (GB)

(21) Appl. No.: 17/759,222

(22) PCT Filed: Jan. 21, 2021
(86) PCT No.: PCT/GB2021/050128

§ 371 Date: Jul. 21, 2022

(30) Foreign Application Priority Data
Jan. 22, 2020 (GB) 2000934.6

(51) Int. Cl. C12N 5/0783; C07K 16/30; C12N 15/86; A61P 35/00; A61K 39/395

(52) U.S. Cl. CPC C12N 5/0636; C07K 16/30; C12N 15/86; A61P 35/00; A61K 39/39558; C07K 2317/732; A61K 2039/505

(57) ABSTRACT

The invention relates to an immune cell that is capable of antibody-dependent cellular cytotoxicity and which comprises a nucleic acid sequence encoding a secreted antigen binding protein. The invention also concerns a method of producing the immune cell and medical uses for the immune cell.

### FIELD OF THE INVENTION

The invention relates to an immune cell that is capable of antibody-dependent cellular cytotoxicity and which comprises a nucleic acid sequence encoding an antigen binding molecule. The invention also concerns a method of producing the immune cell and medical uses for the immune cell.

### BACKGROUND TO THE INVENTION

Alpha beta (ab) T cells expressing chimeric antigen receptors (CARs) form an important part of the immunotherapeutic toolkit. By expressing a CAR in an abT cell, the antigen specificity of the abT cell can be changed. In this way, a subject's adaptive immune system can be reprogrammed to target an antigen of particular interest in the subject, such as a tumour antigen.

Recently, strategies to improve the therapeutic potential of CAR abT cells have been developed. In particular, “armoured” CAR abT cells have been produced. Armoured CAR abT cells are endowed with the ability to secrete function-enhancing molecules. The CAR abT cell may, for instance, secrete a cytokine that enhance its therapeutic effect. CAR abT cells may secrete a peptide which inhibits protein kinase A (Newick et al., Cancer Immunol Res, June 2016, 4(6): 541-551). CAR abT cells may also secrete molecules that overcome the immune checkpoint. For example, Rafiq et al. (Nature Biotechnology, 13 Aug. 2018, 36(9):847-856) equipped CAR abT cells with the ability to secrete an anti-PDL1 antibody, and Li et al. (Clinical Cancer Research, November 2017, 23(22): 6982-6992) engineered CAR abT cells to secrete a scFv specific for PD-1 on the effector cell surface.

In contrast to abT cells, gamma delta (gd) T cells are a relatively overlooked innate-like immune cell subset. Unlike abT cells, gdT cells (especially Vδ2+ gdT cells) are capable of potent antibody-dependent cellular cytotoxicity (ADCC) against antibody-labelled tumour cells. Accordingly, gdT cell infiltration into tumours correlates with favourable clinical outcome and gdT cells have potential in cancer immunotherapy.

Myeloid cells are also capable of ADCC and may be used for cancer immunotherapy. To optimise their therapeutic potential, it would be desirable to engineer gdT cells and myeloid cells to secrete molecules that enhance their anti-cancer effects.

### SUMMARY OF THE INVENTION

The present inventors have demonstrated that immune cells capable of ADCC may be engineered to secrete an antigen binding molecule (e.g. an antibody or antibody-like protein such as a scFv-Fc) targeting an antigen that is expressed in the tumour microenvironment. Secretion of the antigen binding molecule may enhance the anti-cancer effects of the immune cell. For example, the secreted antigen binding molecule may increase target cell killing, for instance by ADCC. This exemplary mechanism is shown in FIG. 1, in which a scFv-Fc secreted by an engineered immune cell (e.g. gdT cell) marks target cells expressing the cognate antigen for ADCC. This allows the engineered immune cells to kill the target cells by ADCC. Bystander, non-engineered immune cells can also exert ADCC against the target cells. In this way, the antigen-specific cytotoxicity of engineered and bystander immune cells is improved. Administration of the engineered immune cells to a subject therefore provides an improved anti-cancer therapy.

Accordingly, the present invention provides:
an immune cell that is capable of ADCC and which comprises a nucleic acid sequence encoding an antigen binding molecule that comprises an antigen binding region;
a method of producing an immune cell of the invention, comprising introducing a nucleic acid sequence encoding an antigen binding molecule into an immune cell;
a method of treating disease in an individual, the method comprising administering to the individual a therapeutically effective number of immune cells of the invention; and
immune cells of the invention for use in a method of treating disease in an individual, the method comprising administering to the individual a therapeutically effective number of the immune cells.

### DESCRIPTION OF THE FIGURES

FIG. 1—Example of a proposed mechanism whereby scFv-Fcs secreted by γδT cells engage Fc receptors on ADCC competent cells in the tumour micro-environment.

FIG. 2—Binding of scFv-Fc to cells expressing the target antigen detected using flow cytometry. CEA+ CAPAN-1 or CEA− HELA cells were incubated with supernatant from Vδ2 cells or Jurkat cells secreting an anti-CEA scFv-Fc fusion protein. Binding of the scFv-Fc fusion protein was detected using anti-human Fc. Purified scFv-fusion protein was used as a positive control. A similar experiment was performed using GD2^{+/-} SupT1 cells and supernatant from Vδ2 cells secreting anti-GD2 scFv-Fc fusion protein. In this case, clinical grade dinutuximab (anti-GD2) was used as a positive control.

FIG. 3—Experimental setup showing conditions used in cytotoxicity assays to test the direct and indirect cytotoxic benefit of scFv-Fc fusion protein expression.

FIG. 4—cytotoxicity in cell-contact dependent and independent settings.

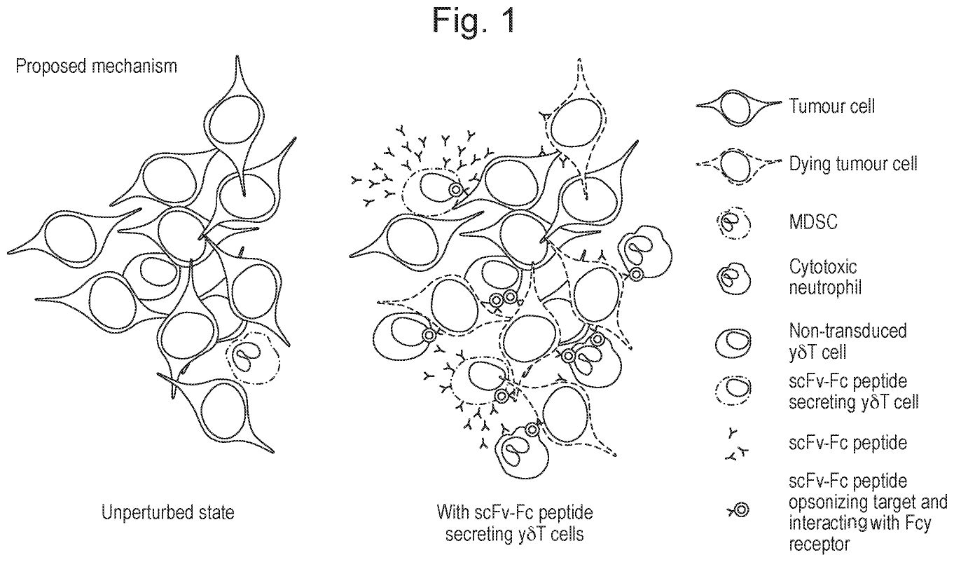

All cytotoxicity experiments were performed at an effector:target ratio of 1:1, using 18 h co-culture. Target cells were labelled with CellTrace Violet™ and death detected by staining with GhostRed fixable viability dye. Target cell death is shown with the background death (in absence of effectors) subtracted from all values.
A) Killing of CEA⁺ CAPAN-1 or CEA− HELA cells by anti-CEA scFv-Fc secreting Vδ2 cells or non-transduced Vδ2 cells.
B) Killing of CEA⁺ CAPAN-1 or CEA− HELA cells by non-transduced Vδ2 cells in the presence or absence of supernatant from anti-CEA-scFv-Fc secreting Vδ2 cells.
C) Killing of CEA⁺ CAPAN-1 or CEA− HELA cells by non-transduced Vδ2 cells. where anti-CEA scFv-Fc secreting Vδ2 cells or non-transduced controls were sequestered behind a semi-permeable membrane.
D) Killing of GD2⁺ SupT1 or GD2⁻ wild type SupT1 cells by anti-GD2 scFv-Fc secreting or non-transduced Vδ2 cells.
E) Killing of GD2⁺ SupT1 or GD2⁻ wild type SupT1 cells by non-transduced Vδ2 cells in the presence or absence of supernatant from anti-GD2-scFv-Fc secreting Vδ2 cells.
F) Killing of GD2⁺ SupT1 or GD2⁻ wild type SupT1 cells by non-transduced Vδ2 cells where anti-GD2 scFv-Fc secreting Vδ2 cells or non-transduced controls were sequestered behind a semi-permeable membrane.

FIG. 5—Concentrations of IFNγ in supernatant after 18 h co-culture of CEA⁺ CAPAN-1 or CEA⁻ HELA cells with non-transduced Vδ2 cells where anti-CEA scFv-Fc secreting Vδ2 cells or non-transduced controls were sequestered behind a semi-permeable membrane.

FIG. 6—Binding of anti-GD2 antibody (SEQ ID NO: 17) produced by 293T cells to GD2^{+/-} target cells detected by flow cytometry. 293T cells were treated with reducing volumes of lentivirus encoding the whole anti-GD2 antibody 14G2a. Isogenic SupT1_wt (GD2⁻) or SupT1_GD2 (GD2⁺) were incubated with supernatant from the transduced 293T cells. Antibody binding was detected using anti-human Fc antibody conjugated to PE. Pure anti-GD2 antibody (dinutuximab, ch14.18, clone 14G2a) was used as a positive control. Antibody was detected in the supernatant of transduced 293T cells, at a level dependent on the viral dose applied.

FIG. 7—Binding of antibody produced by γδT cells to cells expressing target antigen. Isogenic SupT1_wt (GD2⁻) or SupT1_GD2 (GD2⁺) were incubated with supernatant from Vδ2 transduced to express whole anti-Gd2 antibody (clone 14G2a). Antibody binding was detected using anti-human IgG secondary antibody, and pure anti-GD2 (dinutuximab, ch14.18, clone 14G2a) was used as a positive control.

FIG. 8—cytotoxicity in cell-contact dependent and independent settings. All cytotoxicity experiments were performed at an effector:target ratio of 1:1, using 18 h co-culture. Target cells were labelled with CellTrace Violet™ and death detected by staining with Live/Dead Blue fixable viability dye (detected on the DAPI channel). Live/Dead Blue staining of the target cells is shown and dead cell percentages are marked

A) Experimental setup showing how direct and bystander cytotoxicity was determined.
B) Killing of GD2⁺ SupT1 or GD2⁻ wild type SupT1 cells by anti-GD2 antibody secreting or non-transduced Vδ2 cells.
C) Killing of GD2⁺ SupT1 or GD2⁻ wild type SupT1 cells by non-transduced Vδ2 cells in the presence or absence of supernatant from anti-GD2-antibody secreting Vδ2 cells.

### DESCRIPTION OF THE SEQUENCE LISTING

SEQ ID NO: 1 provides the sequence of the V_{H} domain of a CEA-specific scFv used in the examples.

SEQ ID NO: 2 provides the sequence of the V_{L} domain of a CEA-specific scFv used in the examples.

SEQ ID NO: 3 provides the sequence of a CEA-specific scFv-Fc used in the examples.

SEQ ID NO: 4 provides the sequence of the V_{H} domain of a GD2-specific scFv used in the examples.

SEQ ID NO: 5 provides the sequence of the V_{L} domain of a GD2-specific scFv used in the examples.

SEQ ID NO: 6 provides the sequence of a GD2-specific scFv-Fc used in the examples.

SEQ ID NO: 7 provides the sequence of a CEA-specific scFv used in the examples SEQ ID NO: 8 provides the sequence of a GD2-specific scFv used in the examples SEQ ID NO: 9 provides the sequence of the V_{H} domain of a B7H3-specific scFv.

SEQ ID NO: 10 provides the sequence of the V_{L} domain of a B7H3-specific scFv.

SEQ ID NO: 11 provides the sequence of a B7H3-specific scFv-Fc.

SEQ ID NO: 12 provides the sequence of a B7H3-specific scFv.

SEQ ID NO: 13 provides the sequence of the V_{H} domain of a CD20-specific scFv.

SEQ ID NO: 14 provides the sequence of the V_{L} domain of a CD20-specific scFv.

SEQ ID NO: 15 provides the sequence of a CD20-specific scFv-Fc.

SEQ ID NO: 16 provides the sequence of a CD20-specific scFv.

SEQ ID NO: 17 provides the sequence of a GD2 specific IgG1 used in the examples, having a cleavage site between the light and heavy chains.

SEQ ID NO: 18 provides the sequence of the light chain of the GD2 IgG1 of SEQ ID NO: 17.

SEQ ID NO: 19 provides the sequence of the cleavage sequence (Furin-V5-SG-P2A) of the GD2 IgG1 of SEQ ID NO: 17.

SEQ ID NO: 20 provides the sequence of the heavy chain of the GD2 IgG1 of SEQ ID NO: 17.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural referents unless the content clearly dictates otherwise. Thus, for example, reference to “a nucleic acid” includes “nucleic acids”, reference to “an scFv-Fc” includes two or more such scFv-Fcs, reference to “a T cell” includes two or more such T cells, and the like.

All publications, patents and patent applications cited herein, whether supra or infra, are hereby incorporated by reference in their entirety.

### Immune Cells

The invention provides an immune cell that is capable of ADCC and which comprises a nucleic acid sequence encoding an antigen binding molecule. The immune cell may be any immune cell that is capable of ADCC. Immune cells capable of ADCC are known in the art.

ADCC is a well-known mechanism of adaptive, cell-mediated immunity. During ADCC, an immune effector cell actively lyses a target cell whose surface antigens have been bound by specific antibodies. Specifically, antibodies bind their cognate antigen on the surface of target cells. Fc receptors present on the surface of immune effector cells recognise the Fc region of the bound antibodies. Cross-linking of Fc receptors triggers the formation of a lytic synapse between the immune effector cell and the target cell, into which the immune effector cell degranulates lytic granules. Apoptosis of the target cell is therefore triggered. Immune effector cells capable of ADCC are known to include natural killer (NK) cells, macrophages, neutrophils and eosinophils. gdT cells are also capable of ADCC.

The immune cell may be from any species, such as a human, dog, cat, mouse, rat, pig, sheep, cow, goat or horse. The immune cell is typically a human immune cell. The immune cell may be a canine, feline, murine, porcine, ovine, caprine, bovine or equine immune cell.

Preferably, the immune cell is not an abT cell. abT cells are T cells that possess a T cell receptor (TCR) that comprises an alpha chain and a beta chain. They are usually activated in an MHC-dependent manner. ADCC has not been reported for abT cells. abT cells are often thought of as “conventional” T cells.

The immune cell may be a gdT cell. gdT cells are T cells that have a gd T cell receptor (TCR) on their surface. That is, gdT cells possess a TCR that comprises a gamma chain and a delta chain. Therefore, gdT cells are structurally different from abT cells. gdT cells are also functionally different from abT cells. In particular, gdT cells are capable of ADCC. gdT cells are usually activated in an MHC-independent matter. gdT cells are often thought of as “unconventional” T cells.

Several subsets of gdT cell exist. For example, the gdT cell may be a Vδ2+ gdT cell, a Vδ1+ gdT cell, or a Vδ1−/ Vδ2− gdT cell. Preferably, the gdT cell is a Vδ2+ gdT cell. Vδ2+ gdT cells, Vδ1+ gdT cells, and Vδ1−/Vδ2− T cells all have an excellent capacity for ADCC and exhibit good anti-tumour toxicity.

Methods for expanding gdT cells are known in the art. For instance, gdT cells may be expanded by culturing in the presence of IL-2 and zoledronic acid (Fisher J et al.. Effective combination treatment using anti-GD2 ch14.18/CHO antibody with Vδ2+γδT cells in Ewing sarcoma and neuroblastoma. Oncoimmunology 2015 Apr 27;5(1):e1025194.). gdT cells are thus readily available for use in the invention.

The immune cell may be a myeloid cell. Myeloid cells are cells that arise from a common myeloid progenitor cell, such as platelets, erythrocytes, mast cells, macrophages, basophils, neutrophils, and eosinophils. ADCC has been reported for macrophages, basophils, neutrophils, and eosinophils. Preferably, therefore, the myeloid cell is a macrophage, basophil, neutrophil or eosinophil. Methods for isolating and expanding myeloid cells are known in the art.

Natural killer (NK) cells are also capable of ADCC. The immune cell may be a NK cell. NK cells are a class of innate lymphocytes with roles in immunity against a variety of diseases. For example, NK cells have roles in detecting and controlling cancer, and in killing virally-infected cells. Methods for isolating and expanding NK cells are known in the art.

Preferably, the immune cell does not express a chimeric antigen receptor (CAR). Preferably, therefore, the immune cell is not a CAR T cell.

### Nucleic Acid Sequence

The immune cell of the invention comprises a nucleic acid sequence encoding an antigen binding molecule. The nucleic acid sequence may comprise DNA. The nucleic acid sequence may comprise RNA. The nucleic acid sequence may comprise DNA and RNA.

The immune cell may comprise one or more nucleic acid sequences each encoding an antigen binding molecule. For example, the immune cell may comprise 2 or more, 3 or more, 4 or more, 5 or more, 6 or more, 7 or more, 8 or more, 9 or more, 10 or more, 15 or more, or 20 or more nucleic acid sequences each encoding as antigen binding molecule. If the immune cell comprises multiple nucleic acid sequences each encoding an antigen binding molecule, the antigen binding molecule encoded by each nucleic acid sequence may be the same or different. Preferably, each of the antigen binding molecules is different. Preferably, each of the antigen binding molecules is specific for a different antigen. Preferably, the antigen is a tumour antigen. The antigen may be expressed on or by a cancer cell. The antigen may be expressed on or by a non-cancer cell in the tumour microenvironment. The antigen may be secreted into the tumour microenvironment.

Each nucleic acid sequence may encode one or more antigen binding molecules. For example, the nucleic acid sequence may encode 2 or more, 3 or more, 4 or more, 5 or more, 6 or more, 7 or more, 8 or more, 9 or more, 10 or more, 15 or more, or 20 or more antigen binding molecules. If the nucleic acid sequence encodes multiple antigen binding molecules, each of the antigen binding molecules may be the same or different. Preferably, each of the antigen binding molecules is different. Preferably, each of the antigen binding molecules is specific for a different antigen. Preferably, the antigen is a tumour antigen. The antigen may be expressed on or by a cancer cell. The antigen may be expressed on or by a non-cancer cell in the tumour microenvironment. The antigen may be secreted into the tumour microenvironment.

The nucleic acid sequence may comprise an exogenous promoter sequence to which the sequence encoding the antigen binding molecule is operably linked. The exogenous promoter may be an inducible promoter. Alternatively, the nucleic acid sequence may lack an exogenous promoter sequence. In this case, the nucleic acid sequence may integrate to the genome of the immune cell such that expression of the antigen binding molecule is controlled by an endogenous promoter in the genome. Activation of the exogenous or endogenous promoter may be controlled by an inducible signalling pathway. For example, the exogenous or endogenous promoter may be activated following engagement of a synNotch receptor with cognate antigen.

The nucleic acid sequence may be integrated to the genome of the immune cell. Alternatively, the nucleic acid sequence may not be integrated to the genome of the immune cell. If the nucleic acid sequence is not integrated to the genome of the immune cell, it may be comprised in a plasmid, a vector, or an artificial chromosome. The vector may be a viral vector or a non-viral vector. The artificial chromosome may be a yeast artificial chromosome (YAC), a bacterial artificial chromosome (BAC) or a human artificial chromosome (HAC). Preferably, the artificial chromosome is a HAC.

### Antigen Binding Molecule

The immune cell of the invention comprises a nucleic acid sequence encoding an antigen binding molecule.

The antigen binding molecule comprises an antigen binding region. An antigen binding region is a region of an antigen binding molecule that is capable of specifically binding to one or more antigens. For example, an antigen binding region may be capable of specifically binding to 2 or more, 3 or more, 4 or more, 5 or more, 6 or more, 7 or more, 8 or more, 9 or more or 10 or more different antigens. Exemplary antigen binding regions are known in the art, and include at least a scFv (single chain variable fragment), a Fab, a modified Fab, a Fab′, a modified Fab′, a F(ab′)2, a Fv, a dAb, a Fd, a dsFv, a ds-scFv, a scFv2, a Bi-specific T-cell engager, a nanobody, a DARPin, an antibody mimetic, a diabody, a triabody and a tetrabody. The antibody binding region may therefore comprise a scFv, a Fab, a modified Fab, a Fab′, a modified Fab′, a F(ab′)2, a Fv, a dAb, a Fd, a dsFv, a ds-scFv, a scFv2, a Bi-specific T-cell engager, a nanobody, a DARPin, an antibody mimetic, a diabody, a triabody or a tetrabody, alone or in any combination. The antigen binding molecule may comprise a scFv, a Fab, a modified Fab, a Fab′, a modified Fab′, a F(ab′)2, a Fv, a dAb, a Fd, a dsFv, a ds-scFv, a scFv2, a Bi-specific T-cell engager, a nanobody, a DARPin, an antibody mimetic, a diabody, a triabody, a tetrabody, or a polypeptide ligand for a receptor expressed on the surface of a cell that is targeted by the immune cell, alone or in any combination.

Preferably, the antigen binding molecule comprises an antigen binding region comprising a scFv. The antigen binding region may comprise 2 or more, such as 3 or more, 4 or more, 5 or more, 6 or more, 7 or more 8, or more, 9 or more or 10 or more scFvs. scFvs are known in the art. An scFv is a fusion protein comprising the variable region of the heavy chain (V_{H}) tethered to the variable region of the light chain (V_{L}) of an antibody. Typically, the V_{H} and the V_{L} are tethered by a linker peptide. The linker peptide may be from about 5 to about 30 amino acids in length. For example, the linker peptide may be from about 6 to about 29, about 7 to about 28, about 8 to about 27, about 9 to about 26, about 10 to about 25, about 11 to about 24, about 12 to about 23, about 13 to about 22, about 14 to about 21, about 15 to about 20, about 16 to about 19, about 17 or about 18 amino acids in length.

The antigen binding molecule may be capable of binding to a Fc receptor. For example, the antigen binding molecule may comprise a Fc (fragment crystallisable) region.

Fc regions are known in the art. The Fc region is the tail region of an antibody that interacts with Fc receptors and some proteins of the complement system. This property allows antibodies to activate the immune system. The Fc region comprises at least two heavy chain constant (CH) domains. Specifically, in Fc domains derived from an IgG, IgA or an IgD antibody, the Fc region comprises the CH2 and CH3 domains of the antibody. In Fc regions derived from an IgM or an IgE antibody, the Fc region comprise the CH2, CH3 and CH4 regions of the antibody. The Fc region may be a modified Fc region. For example, the Fc region may be an Fc region that has been modified to optimise its ability to bind to FcR. Such optimisation is known in the art and is described, for example in (i) Mossner E et al. Increasing the efficacy of CD20 antibody therapy through the engineering of a new type II anti-CD20 antibody with enhanced direct and immune effector cell-mediated B-cell cytotoxicity. Blood. 2010; 115(22):4393-4402; and (ii) Wang et al. 2018 IgG Fe engineering to modulate antibody effector functions. Protein Cell. 2018 January; 9(1): 63-73. doi: 10.1007/si3238-017-0473-8 (and references therein).

The antigen binding molecule may be capable of binding to a Fc receptor via a region other than a Fc region. That is, the antigen binding molecule need not comprise a Fc region in order to be capable of binding to a Fc receptor. For example, the antigen binding molecule may contain an antigen binding region (such as a scFv, a Fab, a modified Fab, a Fab′, a modified Fab′, a F(ab′)2, a Fv, a dAb, a Fd, a dsFv, a ds-scFv, a scFv2, a Bi-specific T-cell engager, a nanobody, a DARPin, an antibody mimetic, a diabody, a triabody, a tetrabody, or a polypeptide ligand for a receptor expressed on the surface of a cell that is targeted by the immune cell) that is capable of binding to a Fc receptor. The antigen binding molecule may comprise an antigen binding region that is capable of binding to a Fc receptor, and an antigen binding region that is capable of binding to a different antigen, such as an antigen that is expressed in the tumour microenvironment.

For instance, the antigen binding molecule may comprise a scFv that is capable of binding to a Fc receptor, and a scFv that is capable of binding to a different antigen, such as an antigen that is expressed in the tumour microenvironment. The antigen binding molecule may comprise a bi-specific T-cell engager the comprises a scFv that is capable of binding to CD3, and a scFv that is capable of binding to a different antigen, such as an antigen that is expressed in the tumour microenvironment.

The antigen binding molecule may comprise an antigen binding region and a Fc region. The antigen binding molecule may be an antibody, a scFv-Fc, a dAb-Fc, or a heavy chain antibody. Exemplary heavy chain antibodies include an IgNAR and a camelid antibody. The antibody may, for example, comprise (a) a light chain encoded by SEQ ID NO: 18, (b) a heavy chain encoded by SEQ ID NO: 20, and/or (c) a cleavage sequence encoded by SEQ ID NO: 19. The antibody molecule may, for example, comprise: (a); (b); (c); (a) and (b); (a) and (c); (b) and (c); or (a), (b) and (c). The antibody may be encoded by SEQ ID NO: 17. The light chain encoded by SEQ ID NO: 18 comprises the V_{L} encoded by SEQ ID NO: 5. The heavy chain encoded by SEQ ID NO: 20 comprises the V_{H} encoded by SEQ ID NO:4.

Preferably, the antigen binding molecule is a scFv-Fc. An scFv-Fc is a fusion protein that comprises a scFv fused to a Fc region. The structure of scFvs and the Fc region is known in the art and described above. In accordance with these structures, the scFv-Fc may comprise a V_{H} and a V_{L} (together forming the scFv) and a CH2 domain and a CH3 domain (together forming the Fc region). The scFv-Fc may comprise a V_{H} and a V_{L} (together forming the scFv), and a CH2 domain, a CH3 domain and a CH4 domain (together forming the Fc region). In a scFv-Fc, the scFv is linked to the Fc region.

Preferably, the V_{L} or the V_{H} in the scFv is linked to the CH2 in the Fc region in order to link the scFv to the Fc region. The scFv may be linked to the Fc region directly, i.e. in the absence of a linker. The scFv may be linked to the Fc region by a linker. The linker may be (Ser(Gly)₄), (Ser(Gly)₄)₂, (Ser(Gly)₄)₃, (Ser(Gly)₄)₄, or (Ser(Gly)₄)₅. The linker may be an amino acid, or a short oligopeptide, consisting of about 2 amino acids. The linker may form a hinge region. The scFv-Fc may be a bivalent scFv-Fc. A bivalent scFv-Fc comprises two different scFvs each linked to a Fc region. In essence, a bivalent scFv-Fc comprises two arms, each comprising a scFv linked to a Fc region. scFvs, Fc regions and linkage are discussed above. The two arms are preferably linked. Linkage between the two arms preferably connects a linker between the scFv and Fc region in one arm with a linker between the scFv and Fc region in the other arm. The two arms are preferably connected at a point that is between the scFv and the Fc region in each arm.

The antigen binding molecule may function to increase target cell cytotoxicity. That is, the antigen binding molecule may increase the killing of cells expressing the cognate antigen of the antigen binding region. For example, the antigen binding molecule may increase the killing of tumour cells, endothelial cells, and/or immune cells. Increased killing may be mediated by engineered immune cells (i.e. by immune cells comprising the nucleic acid sequence encoding an antigen binding molecule). Increased killing may be mediated by non-engineered (“bystander”) immune cells (i.e. by immune cells that do not comprise the nucleic acid sequence encoding an antigen binding molecule). Increased killing may be mediated by both engineered immune cells and non-engineered immune cells. Increased killing may be by any mechanism known in the art. Preferably, increased killing is mediated by increased ADCC. Increased killing may be mediated by increased engagement of ab T cells.

The antigen binding molecule is preferably an opsonin. An opsonin is a molecule that binds to an antigen to enhance its phagocytosis. Binding of an opsonin to an antigen may favour interactions between the antigen and cell surface receptors on immune cells, thereby boosting the kinetics of phagocytosis. Accordingly, the antigen binding molecule may enhance phagocytosis. In particular, the antigen binding molecule may enhance phagocytosis of an antigen for which the antigen binding region is specific. In other words, the antigen binding molecule may enhance phagocytosis of an antigen bound by the antigen binding region (and, therefore, the antigen binding molecule).

The antigen binding region (and the antigen binding molecule) may be capable of binding to any antigen. That is, any antigen may be bound by the antigen binding region (and the antigen binding molecule). Preferably, the antigen binding region (and the antigen binding molecule) is capable of binding to an antigen that is expressed on or by cells in the tumour microenvironment. An antigen is expressed in the tumour microenvironment if it is expressed by any type of cell present in the tumour microenvironment. For example, the antigen may be expressed by a tumour cell, an endothelial, or an immune cell in the tumour microenvironment. The antigen binding region (and the antigen binding molecule) may therefore be capable of binding to a tumour antigen, an endothelial antigen, or an immune cell antigen. The immune cell may, for example, be a CD4+ T cell, a CD8+ T cell, a gdT cell, a B cell, a NK cell, a NKT cell, a macrophage, a monocyte, a basophil, an eosinophil or a neutrophil.

The antigen binding region (and the antigen binding molecule) may be capable of binding to an antigen selected from a group consisting of TSHR, CD19, CD123, CD22, CD20, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7-H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp1OO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OYTES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LTLRA2, CD300LF, CLEC12A, BST2, EMR2, LY75, GPC3, FCRL5, CEA, LINGO1, CD70, IL13Ra2, MUC-16, PSCA, ROR1, and IGLL1.

The antigen binding region (and antigen binding molecule) may be capable of binding to carcinoembryonic antigen (CEA). The CEA may be CEA-CAM5. CEA may be expressed in many carcinomas, including those of the colon and/or rectum, stomach, breast, pancreas, lung, thyroid, uterine cervix, or ovary.

The CEA-specific antigen binding region (and antigen binding molecule) may comprise a V_{H} domain encoded by SEQ ID NO: 1. The CEA-specific antigen binding region (and antigen binding molecule) may comprise a V_{L} domain encoded by SEQ ID NO: 2. The CEA-specific antigen binding region (and antigen binding molecule) may comprise a V_{H} domain encoded by SEQ ID NO: 1 and a V_{L} domain encoded by SEQ ID NO: 2. The CEA-specific antigen binding region (and antigen binding molecule) may comprise complementarity determining regions (CDRs) from a V_{H} domain encoded by SEQ ID NO: 1 and/or a V_{L} domain encoded by SEQ ID NO: 2. That is, the CEA-specific antigen binding region (and antigen binding molecule) may comprise heavy chain CDR1, CDR2 and/or CDR3 from a V_{H} domain encoded by SEQ ID NO: 1. The CEA-specific antigen binding region (and antigen binding molecule) may comprise light chain CDR1, CDR2 and/or CDR3 from a V_{L} domain encoded by SEQ ID NO: 2. The CEA-specific antigen binding region (and antigen binding molecule) may comprise heavy chain CDR1, CDR2 and/or CDR3 from a V_{H} domain encoded by SEQ ID NO: 1 and light chain CDR1, CDR2 and/or CDR3 from a V_{L} domain encoded by SEQ ID NO: 2. The CEA-specific antigen binding molecule may be a scFv-Fc encoded by SEQ ID NO: 3. The scFv may have the amino acid sequence of SEQ ID NO: 7.

The antigen binding region (and antigen binding molecule) may be capable of binding to GD2. GD2 may be expressed by cancers of neuroectodermal origin, such as neuroblastoma and melanoma.

The GD2-specific antigen binding region (and antigen binding molecule) may comprise a V_{H} domain encoded by SEQ ID NO: 4. The GD2-specific antigen binding region (and antigen binding molecule) may comprise a V_{L} domain encoded by SEQ ID NO: 5. The GD2-specific scFv-Fc antigen binding region (and antigen binding molecule) may comprise a V_{H} domain encoded by SEQ ID NO: 4 and a V_{L} domain encoded by SEQ ID NO: 5. The GD2-specific antigen binding region (and antigen binding molecule) may comprise CDRs from a V_{H} domain encoded by SEQ ID NO: 4 and/or a V_{L} domain encoded by SEQ ID NO: 5. That is, the GD2⁻ antigen binding region (and antigen binding molecule) may comprise heavy chain CDR1, CDR2 and/or CDR3 from a V_{H} domain encoded by SEQ ID NO: 4. The GD2-specific antigen binding region (and antigen binding molecule) may comprise light chain CDR1, CDR2 and/or CDR3 from a V_{L} domain encoded by SEQ ID NO: 5. The GD2-specific antigen binding region (and antigen binding molecule) may comprise heavy chain CDR1, CDR2 and/or CDR3 from a V_{H} domain encoded by SEQ ID NO: 4 and light chain CDR1, CDR2 and/or CDR3 from a V_{L} domain encoded by SEQ ID NO: 5. The GD2-specific antigen binding molecule may be a scFv-Fc encoded by SEQ ID NO: 6. The scFv may have the amino acid sequence of SEQ ID NO: 8.

The GD2-specific antigen binding molecule may comprise (a) a light chain encoded by SEQ ID NO: 18, (b) a heavy chain encoded by SEQ ID NO: 20, and/or (c) a cleavage sequence. The GD2-specific antigen binding molecule may, for example, comprise: (a); (b); (c); (a) and (b); (a) and (c); (b) and (c); or (a), (b) and (c). Preferably, the GD2-specific antigen binding molecule comprises (a), (b) and (c) in that order. In any of the aspects described herein, the cleavage sequence (c) may, for example, be Furin-V5-SG-P2A. The cleavage sequence may, for example, be encoded by SED ID NO: 19. Other cleavage sequences are also known in the art and may be used as the cleavage sequence (c). For example, the cleavage sequence (c) may comprise or consist of P2A, E2A, F2A or T2A. An IRES or IRES 2 sequence may be used in place of the cleavage sequence (c), in any aspect described herein. The GD2-specific antigen binding molecule may be an antibody encoded by SEQ ID NO: 17.

The antigen binding region (and antigen binding molecule) may be capable of binding to B7-H3. B7-H3 may be expressed by cancers of neuroectodermal origin, such as neuroblastoma and melanoma.

The B7-H3-specific antigen binding region (and antigen binding molecule) may comprise a V_{H} domain encoded by SEQ ID NO: 9. The B7-H3-specific antigen binding region (and antigen binding molecule) may comprise a V_{L} domain encoded by SEQ ID NO: 10. The B7-H3-specific antigen binding region (and antigen binding molecule) may comprise a V_{H} domain encoded by SEQ ID NO: 9 and a V_{L} domain encoded by SEQ ID NO: 10. The B7-H3-specific antigen binding region (and antigen binding molecule) may comprise CDRs from a V_{H} domain encoded by SEQ ID NO: 9 and/or a V_{L} domain encoded by SEQ ID NO: 10. That is, the B7-H3-specific antigen binding region (and antigen binding molecule) may comprise heavy chain CDR1, CDR2 and/or CDR3 from a V_{H} domain encoded by SEQ ID NO: 9. The B7-H3-specific antigen binding region (and antigen binding molecule) may comprise light chain CDR1, CDR2 and/or CDR3 from a V_{L} domain encoded by SEQ ID NO: 10. The B7-H3-specific antigen binding region (and antigen binding molecule) may comprise heavy chain CDR1, CDR2 and/or CDR3 from a V_{H} domain encoded by SEQ ID NO: 9 and light chain CDR1, CDR2 and/or CDR3 from a V_{L} domain encoded by SEQ ID NO: 10. The B7-H3-specific antigen binding molecule may be a scFv-Fc encoded by SEQ ID NO: 11. The scFv may have the amino acid sequence of SEQ ID NO: 12.

The antigen binding region (and antigen binding molecule) may be capable of binding to CD20. CD20 is expressed during B cell development, the late pro-B cell stage through memory cells (though not on early pro-B cells or plasma blasts and plasma cells). CD20 is also expressed in B-cell lymphomas, hairy cell leukemia, B-cell chronic lymphocytic leukemia, and melanoma cancer stem cells.

The CD20-specific antigen binding region (and antigen binding molecule) may comprise a V_{H} domain encoded by SEQ ID NO: 13. The CD20-specific antigen binding region (and antigen binding molecule) may comprise a V_{L} domain encoded by SEQ ID NO: 14. The CD20-specific antigen binding region (and antigen binding molecule) may comprise a V_{H} domain encoded by SEQ ID NO: 13 and a V_{L} domain encoded by SEQ ID NO: 14. The CD20-specific antigen binding region (and antigen binding molecule) may comprise CDRs from a V_{H} domain encoded by SEQ ID NO: 13 and/or a V_{L} domain encoded by SEQ ID NO: 14. That is, the CD20-specific antigen binding region (and antigen binding molecule) may comprise heavy chain CDR1, CDR2 and/or CDR3 from a V_{H} domain encoded by SEQ ID NO: 13. The CD20-specific antigen binding region (and antigen binding molecule) may comprise light chain CDR1, CDR2 and/or CDR3 from a V_{L} domain encoded by SEQ ID NO: 14. The CD20-specific antigen binding region (and antigen binding molecule) may comprise heavy chain CDR1, CDR2 and/or CDR3 from a V_{H} domain encoded by SEQ ID NO: 13 and light chain CDR1, CDR2 and/or CDR3 from a V_{L} domain encoded by SEQ ID NO: 14. The CD20-specific antigen binding molecule may be a scFv-Fc encoded by SEQ ID NO: 15. The scFv may have the amino acid sequence of SEQ ID NO: 16.

The antigen binding molecule is preferably expressed by the immune cell that comprises the nucleic acid sequence encoding the antigen binding molecule. Expression of the antigen binding molecule may be determined based on the presence of mRNA encoding the antigen binding molecule in the immune cell. Preferably, expression of the antigen binding molecule is determined based on the presence of the antigen binding molecule itself in the immune cell. Methods for determining the presence of a mRNA or a protein in a cell are well-known in the art. For example, reverse transcriptase PCR or Northern blotting may be used to determine the presence of an mRNA in a cell. Flow cytometry, immunofluorescent imaging or western blotting may be used to determine the presence of a protein in a cell.

### Method of Producing an Immune Cell

The invention provides a method of producing an immune cell of the invention. The method comprises introducing a nucleic acid sequence encoding an antigen binding molecule into an immune cell. Antigen binding molecules are described in detail above. The nucleic acid sequence may, for example, be introduced to the immune cell by transduction or transfection. For example, T-cell transduction methods known in the art may be used to transduce gdT cells may be with the nucleic acid sequence. gdT cells may be expanded prior to such transduction, for example by culturing in the presence of IL-2 and zoledronic acid for a period of about 5 days (such as about 3 days, about 4 days, about 6 days, or about 7 days).

The term “transduction” may be used to describe virus-mediated nucleic acid transfer. A viral vector may be used to transduce the cell with the nucleic acid sequence. Thus, the nucleic acid sequence may be comprised in a viral vector. The viral vector may be a retroviral, lentiviral, adenoviral, adeno-associated (AAV) or herpes simplex virus (HSV) vector. Preferably, the viral vector is a retroviral vector. Methods for producing and purifying such vectors are known in the art. The immune cell may be transduced using any method known in the art. Transduction may be in vitro or ex vivo.

The term “transfection” may be used to describe non-virus-mediated nucleic acid transfer. The immune cell may be transfected using any method known in the art. Transfection may be in vitro or ex vivo. Any vector capable of transfecting the immune cell may be used, such as conventional plasmid DNA or RNA transfection. A human artificial chromosome and/or naked RNA and/or siRNA may be used to transfect the cell with the nucleic acid sequence. Human artificial chromosomes are described in e.g. Kazuki et al., Mol. Ther. 19(9): 1591-1601 (2011), and Kouprina et al., Expert Opinion on Drug Delivery 11(4): 517-535 (2014). Alternative non-viral delivery systems include DNA plasmids, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Nanoparticle delivery systems may be used to transfect the cell with the nucleic acid sequence or nucleic acid construct. Such delivery systems include, but are not limited to, lipid-based systems, liposomes, micelles, microvesicles and exosomes. With regard to nanoparticles that can deliver RNA, see, e.g., Alabi et al., Proc Natl Acad Sci USA. 2013 Aug 6;110(32):12881-6; Zhang et al., Adv Mater. 2013 Sep 6;25(33):4641-5; Jiang et al., Nano Lett. 2013 Mar 13;13(3):1059-64; Karagiannis et al., ACS Nano. 2012 Oct 23;6(10):8484-7; Whitehead et al., ACS Nano. 2012 Aug 28;6(8):6922-9 and Lee et al., Nat Nanotechnol. 2012 Jun 3;7(6):389-93. Lipid Nanoparticles, Spherical Nucleic Acid (SNA™) constructs, nanoplexes and other nanoparticles (particularly gold nanoparticles) are also contemplated as a means for delivery of a construct or vector in accordance with the invention.

Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectAmine, fugene and transfectam.

The immune cell may be transfected under suitable conditions. The immune cell and agent or vector may, for example, be contacted for between five minutes and ten days, preferably from an hour to five days, more preferably from five hours to two days and even more preferably from twelve hours to one day.

The nucleic acid sequence transduced or transfected into the immune cell gives rise to expression of the antigen binding molecule in the immune cell. The nucleic acid sequence preferably comprises a promoter which is operably linked to the sequence encoding the antigen binding molecule. The promoter may be constitutively active in the immune cell. The promoter may be inducible in the immune cell.

### Medicaments, Methods and Therapeutic Use

The immune cells of the invention may be used in a method of therapy of the human or animal body. Thus, the invention provides a method of treating disease in an individual, the method comprising administering to the individual a therapeutic number of immune cells of the invention. The invention further provides immune cells of the invention for use in a method of treating disease in an individual, the method comprising administering to the individual a therapeutic amount of the immune cells The individual may be of species. For example, the individual may be a human, dog, cat, mouse, rat, pig, sheep, cow, goat or horse. Preferably, the individual is human.

Preferably, the immune cells are of the same species as the individual. The immune cells may be autologous with respect to the individual. The immune cells may be allogeneic with respect to the individual. The individual may be an infant, a juvenile or an adult. The individual may have, be susceptible to, or be at risk from, the disease.

The invention concerns administering to the individual a therapeutically effective number of immune cells of the invention. A therapeutically effective number is a number which ameliorates one or more symptoms of the disease. A therapeutically effective number is preferably a number which treats the disease. Any suitable number of immune cells may be administered to the individual. As a guide, the number of immune cells to be administered is typically from 10⁵ to 10⁹, preferably from 10⁶ to 10⁸. For example, at least, or about, 0.2×10⁶, 0.25×10⁶, 0.5×10⁶, 1.5×10⁶, 4.0×10⁶ or 5.0×10⁶ immune cells per kg of individual may administered. For example, at least, or about, 10⁵, 10⁶, 10⁷, 10⁸, or 10⁹ immune cells may be administered. At least about 1×10⁶, at least about 2×10⁶, at least about 2.5 2×10⁶, at least about 5×10⁶, at least about 1×10⁷, at least about 2×10⁷, at least about 5×10⁷, at least about 1×10⁸ or at least about 2×10⁸ immune cells may be administered.

The immune cells may be used in combination with other means of, and substances for, treating the disease. For example, the immune cells may be used in combination with one or more cancer therapies. For example, the immune cells may be used in combination with one or more chemotherapeutic agent. The immune cells may be used in combination with one or more CAR-expressing ab T cells. The immune cells may be used in combination with radiotherapy. The immune cells may be used in combination with surgery, for example surgery to resect or remove a tumour.

The immune cells may be used in combination with one or more therapies for treating an infectious disease, such as a viral infection or a bacterial infection. For example, the immune cells may be used in combination with one or more anti-viral drug. The immune cells may be used in combination with one or more antibiotics.

The immune cells may be used in combination with substances that support immune cell function. For example, the immune cells may be used in combination with an aminobisphosphonate, such as zoledronic acid. The immune cells may be used in combination with an aminobisphosphonate when, for example, the immune cells are Vδ2+ gdT cells. The immune cells may be used in combination with one or more immune stimulating cytokines, such as IL-2, GM-CSF or G-CSF. IL-2, GM-CSF or G-CSF may each enhance other populations of ADCC-competent cells.

When immune cells are used in combination with one or more other substances, immune cells may be administered simultaneously with, sequentially with or separately from the other substance(s). The immune cells may be used in combination with existing treatments for treating the disease and may, for example, be simply mixed with such treatments. Thus, the immune cells may be used to increase the efficacy of existing treatments for the disease.

The immune cells may be formulated for administration using any suitable method. Formulation of cells with standard pharmaceutically acceptable carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19^{th} Edition, Mack Publishing Company, Eastern Pennsylvania, USA.

The immune cells may be formulated with a physiologically acceptable carrier or diluent. Typically, such formulations are prepared as liquid suspensions of cells. The cells may be mixed with an excipient which is pharmaceutically acceptable and compatible with the active ingredient. Suitable excipients are, for example, water, saline, dextrose, glycerol, of the like and combinations thereof. In addition, if desired, the pharmaceutical compositions of the invention may contain minor amounts of auxiliary substances such as wetting or emulsifying agents, pH buffering agents, and/or adjuvants which enhance effectiveness.

The immune cells may be administered by any route. Suitable routes include, but are not limited to, intravenous, intramuscular, intraperitoneal or other appropriate administration routes. The immune cells are preferably administered intravenously.

The disease may be any disease in which the patients may benefit from targeted ADCC. The disease may be any disease in which the patients may benefit from a targeted T-cell response. For instance, the disease may be a disease in which the subject may benefit from the killing of unwanted cells. The unwanted cells may, for example, be cancer cells. The unwanted cells may be or cells infected with bacteria, a virus, a fungus, protozoa, or a parasite. The unwanted cells may be aberrant immune cells, i.e. immune cells that give rise to a detrimental immune response. For example, the aberrant immune cells may be autoimmune cells. Accordingly, the disease may be an infection (such as a bacterial, viral, fungal, protozoal or other parasitic infection) or an autoimmune disease. Preferably, the disease is cancer. The cancer may be a cancer of the haematopoietic tissue and/or lymphoid tissue. Preferably, the cancer is a solid tumour.

The cancer may be primary cancer or secondary cancer. The cancer may be anal cancer, bile duct cancer (cholangiocarcinoma), bladder cancer, blood cancer, bone cancer, bowel cancer, brain tumours, breast cancer, colorectal cancer, cervical cancer, endocrine tumours, eye cancer (such as ocular melanoma), fallopian tube cancer, gall bladder cancer, head and/or neck cancer, Kaposi's sarcoma, kidney cancer, larynx cancer, leukaemia, liver cancer, lung cancer, lymph node cancer, lymphoma, melanoma, mesothelioma, myeloma, neuroendocrine tumours, ovarian cancer, oesophageal cancer, pancreatic cancer, penis cancer, primary peritoneal cancer, prostate cancer, skin cancer, small bowel cancer, soft tissue sarcoma, spinal cord tumours, stomach cancer, testicular cancer, thymus cancer, thyroid cancer, trachea cancer, unknown primary cancer, vagina cancer, vulva cancer or endometrial cancer. The leukaemia may be acute lymphoblastic leukaemia, acute myeloid leukaemia (AMIL), chronic lymphocytic leukaemia or chronic myeloid leukaemia. The lymphoma may be Hodgkin lymphoma or non-Hodgkin lymphoma. The cancer may be neuroblastoma or melanoma. The cancer may be colon cancer, rectal cancer, stomach cancer, breast cancer, lung cancer, thyroid cancer or ovary cancer. The cancer may be a carcinoma. Preferably, the cancer is a colorectal cancer or a neuro-endocrine tumour.

### EXAMPLES

Vδ2⁺ γδT Cells can be Transduced to Secrete Proteins which Specifically Bind to Antigen on Target Cells.

Vδ2⁺γδT cells were transduced to secrete an scFv fusion peptide targeting the tumour associated antigens CEA and GD2. Supernatant from transduced cells was harvested and applied to CEA⁺ CAPAN-1 cells and CEA⁻ HELA cells, or to GD2+^{+/-} SupT1 cells. Binding of scFv-Fc fusion protein was detected using a phycoerythrin conjugated anti-human Fc antibody. Purified anti-CEA scFv-Fc fusion protein or purified anti-GD2 antibody, produced in cell lines were used as a positive controls.

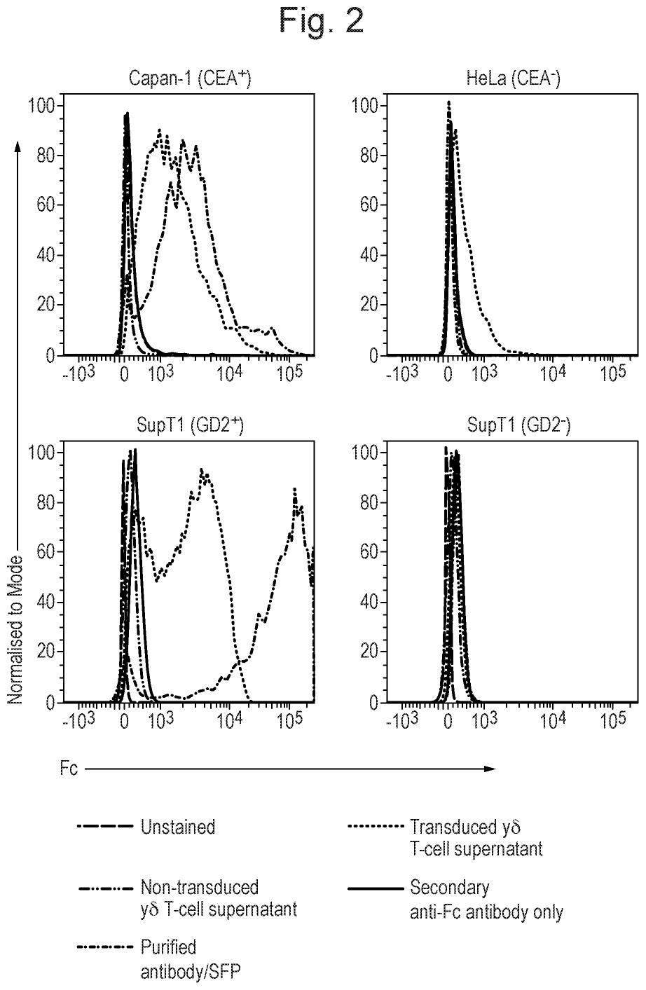

As shown in FIG. 2, a constituent of the supernatant from transduced cell lines and transduced Vδ2γδT cells was able to specifically bind antigen positive cells and led to detection of human Fc on their surface.

Cytotoxicity Experiments Demonstrating the Effects of Direct and Indirect Contact with scFv-Fc Protein Secreting Vδ2

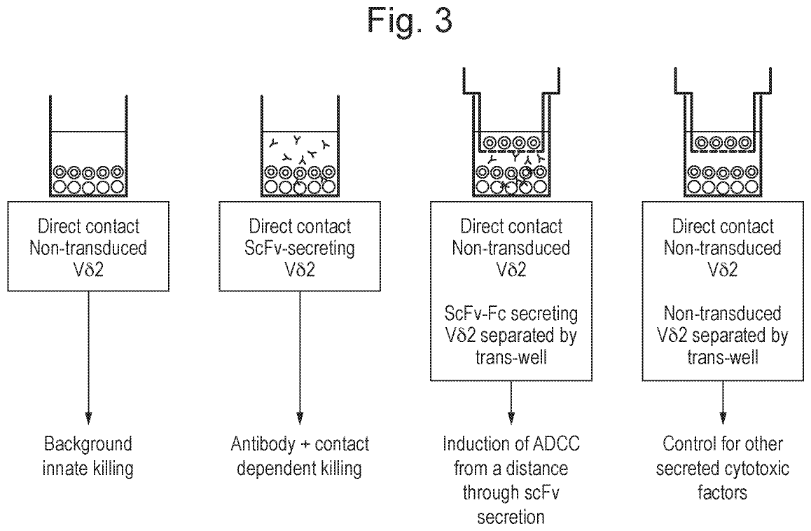

Because the engineered T-cells secrete a protein which may have effects on the cytotoxicity of bystander, non-engineered cells, a series of experimental conditions were developed to demonstrate this. The experimental setup is illustrated in FIG. 3. Briefly, in addition to comparting cytotoxicity of transduced and non-transduced Vδ2 upon direct co-culture with target cells, two conditions were added to demonstrate the cell-contact autonomous effects. Firstly, supernatant from transduced Vδ2 was added to a co-culture of non-transduced Vδ2 and target cells. Secondly, non-transduced cells were co-cultured with targets in the presence of transduced cells which were sequestered behind a semi-permeable membrane which blocks passage of cells but not of secreted molecules. All co-cultures were 18 h, at an effector:target ratio of 1:1. For demonstration of anti-CEA scFv-Fc fusion protein action, CEA⁺ CAPAN-1 and CEA⁻ HELA cells were used. For demonstration of anti-GD2 scFv-Fc fusion protein action, SupT1 engineered to express GD2 or isogenic GD2− wild type SupT1 were used.

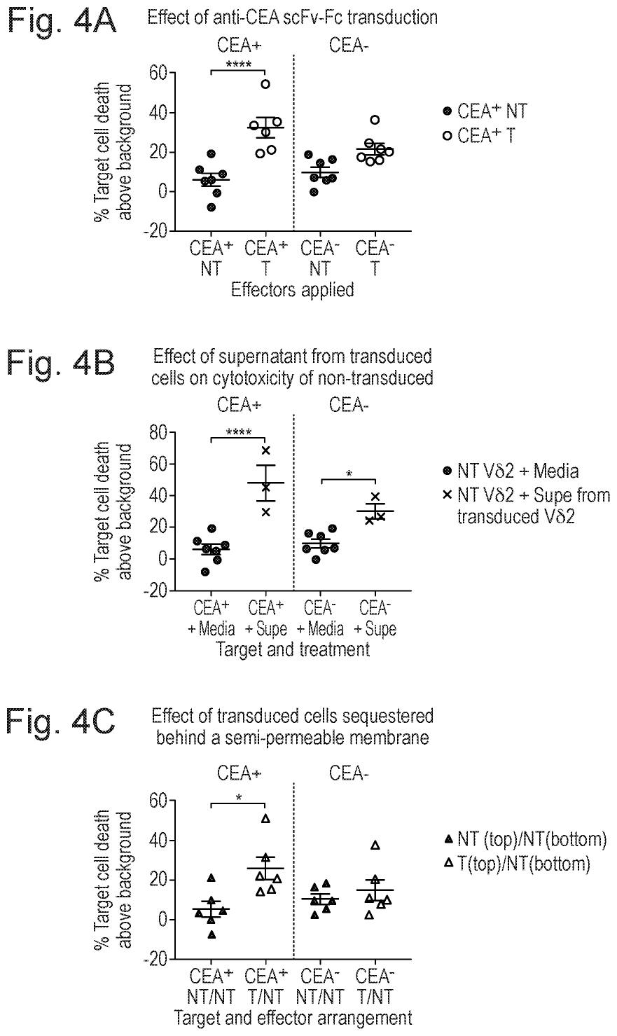

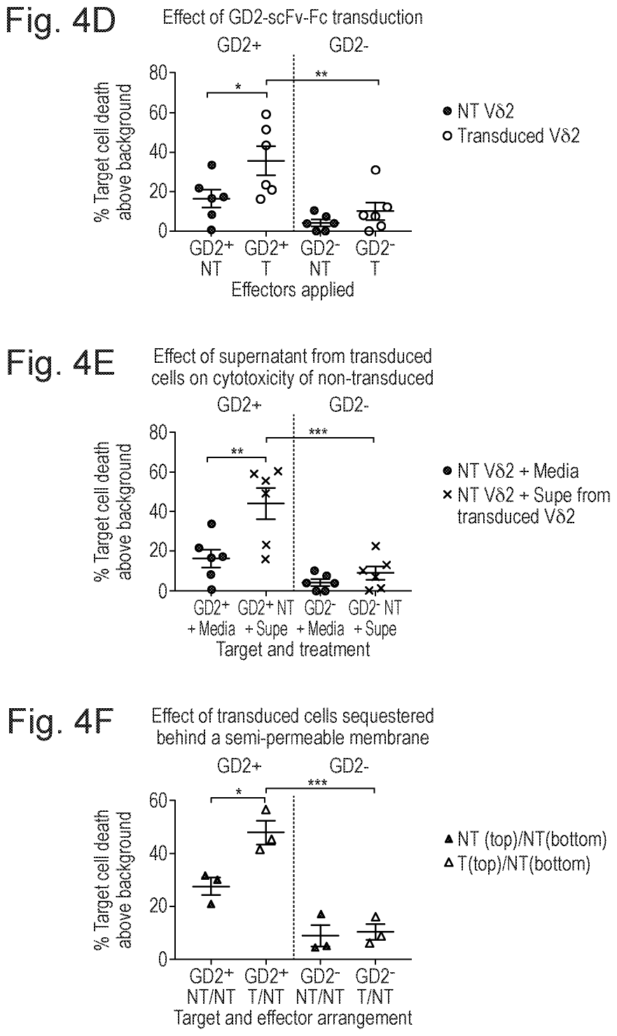

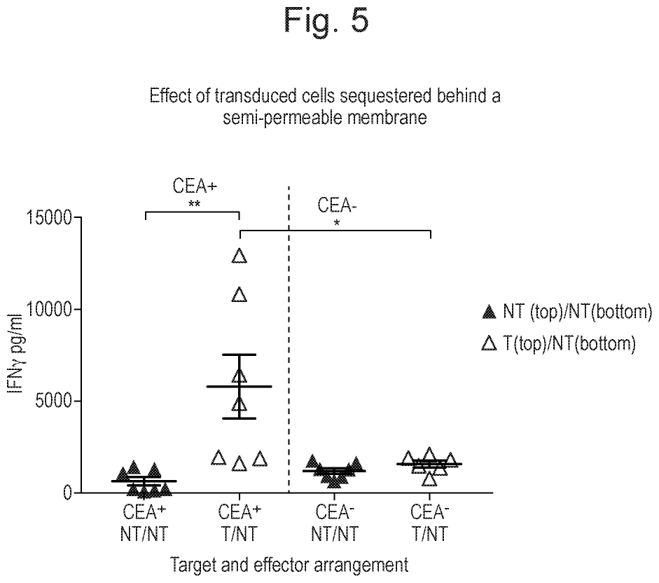

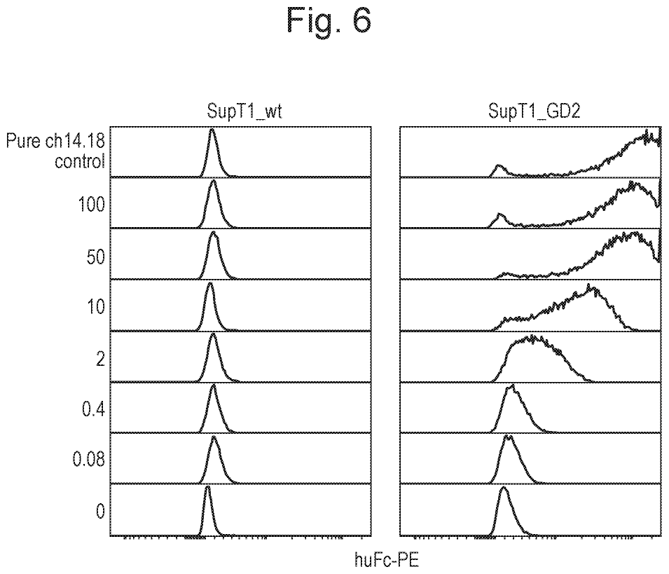

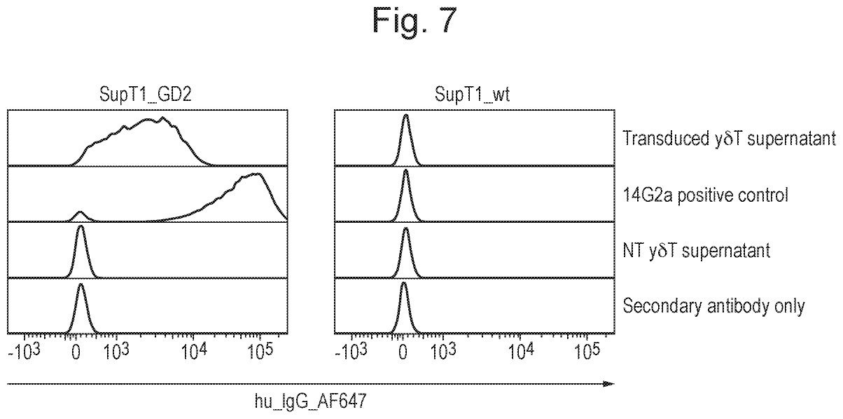

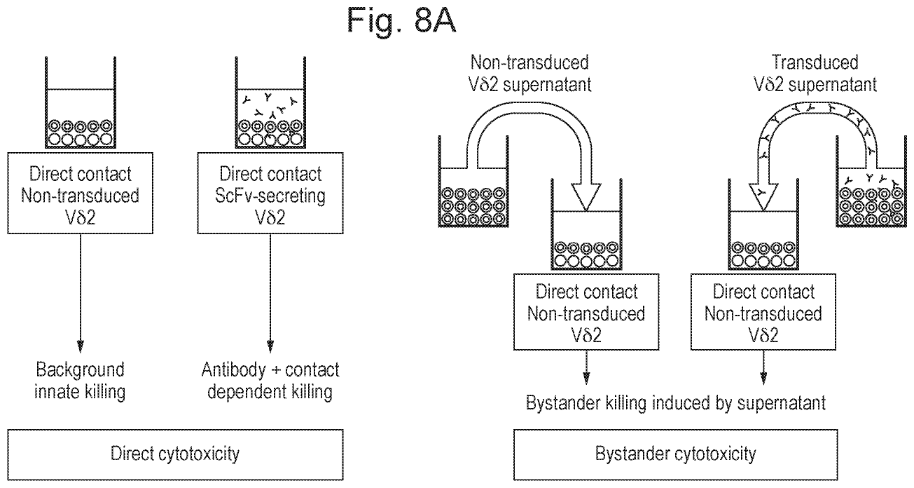

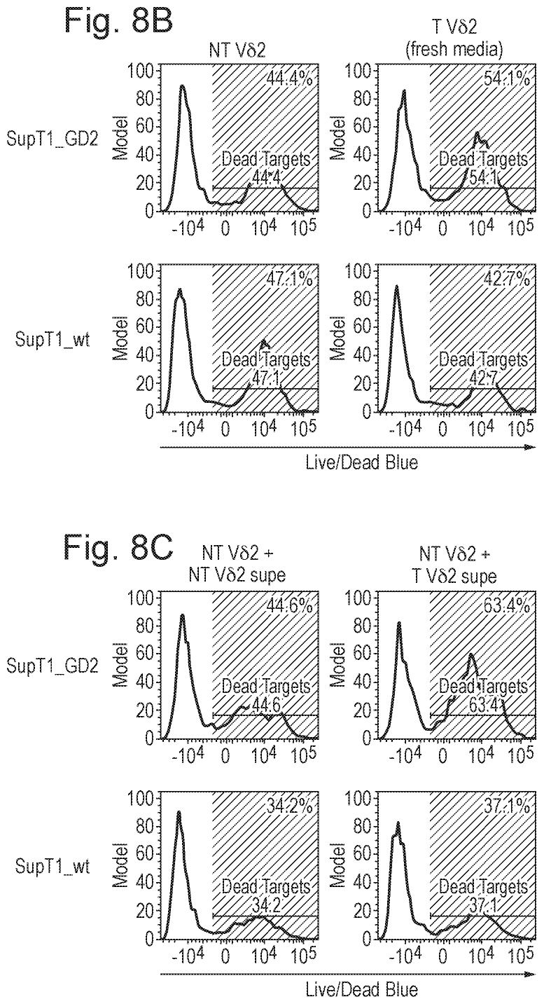

Vδ2 cells transduced to secrete anti-CEA scFv-Fc fusion proteins led to significantly higher death of CEA⁺ targets compared to NT Vδ2 (p<0.0001, FIG. 4A). Supernatant from Vδ2⁺ cells transduced to express anti-CEA scFv-Fc fusion proteins enhanced killing of CEA⁺ targets by non-transduced Vδ2 (p<0.0001) and also led to a small increase in killing of CEA⁻ targets (p=0.031, FIG. 4B). When anti-CEA scFv-Fc fusion protein secreting Vδ2 were sequestered behind a semi-permeable membrane, they were also able to enhance the ability of non-transduced Vδ2 to kill CEA⁺ targets when compared to sequestration of non-transduced cells (p=0.012, FIG. 4C).

Similar data were observed for Vδ2 transduced to secrete an anti-GD2 scFv-Fc fusion protein. Transduced Vδ2 killed GD2⁺ targets better than non-transduced (p=0.03) and exhibited greater cytotoxicity against GD2⁺ targets than isogenic GD2⁻ controls (p=0.0049, FIG. 4D). Supernatant from anti-GD2-scFv-Fc fusion protein expressing Vδ2 significantly enhanced the ability of non-transduced Vδ2 to kill GD2⁺ targets when compared to non-transduced controls (p=0.0025) or isogenic GD2⁻ targets (p=0.0002, FIG. 4E). When anti-GD2 scFv-Fc fusion protein secreting Vδ2 were sequestered behind a semi-permeable membrane, they were also able to enhance the ability of non-transduced Vδ2 to kill GD2⁺ targets when compared to sequestration of non-transduced cells (p=0.0158) or killing of GD2⁻ targets (p=0.0003, FIG. 4F).

When non-transduced Vδ2 were co-cultured with CEA^{±} targets in the presence of anti-CEA scFv-Fc fusion protein secreting Vδ2 or non-transduced controls sequestered behind a semi-permeable membrane, significant increases in IFNγ production were only observed when anti-CEA scFv-Fc fusion protein secreting cells were sequestered (p=0.0014 comparted to sequestered NT Vδ2) and CEA⁺ targets were used (p=0.012 compared to CEA⁻ targets, FIG. 5).

Vδ2⁺ γδT Cells can be Transduced to Secrete Whole Antibody which Specifically Binds to Antigen on Target Cells.

HEK293T and Vδ2⁺γδT cells were transduced to secrete an IgG1 antibody targeting the tumour associated antigen GD2 (SEQ ID NO: 17). Supernatant from HEK293T cells transduced with reducing volumes of virus was harvested and applied to GD2^{+/-} SupT1 cells. Binding of scFv-Fc fusion protein was detected using a phycoerythrin conjugated anti-human Fc antibody (FIG. 6). Supernatant from transduced Vδ2⁺γδT cells was also harvested and applied to GD2^{+/-} SupT1 cells, and antibody binding detected using AlexaFluor 647 conjugated anti-human IgG antibody (FIG. 7). Purified anti-GD2 antibody, produced in cell lines was used as a positive control.

As shown in FIGS. 6 and 7, a constituent of the supernatant from transduced cell lines and transduced Vδ2⁺γδT cells was able to specifically bind antigen positive cells and led to detection of human Fc or IgG on their surface.

Cytotoxicity Experiments Demonstrating the Effects of Direct and Indirect Contact with Antibody Secreting Vδ2

In order to demonstrate the cell-contact dependent and cell-contact autonomous effects, two experimental systems were used (FIG. 8A). Briefly, non-transduced Vδ2⁺γδT cells or Vδ2⁺γδT cells transduced to express anti-GD2 IgG1 were co-cultured at a 1:1 effector:target ratio with GD2+^{+/-} SupT1. In the second experiment, supernatant from non-transduced Vδ2⁺γδT cells or supernatant from Vδ2⁺γδT cells transduced to express anti-GD2 IgG1 was added to a 1:1 co-culture of non-transduced Vδ2⁺γδT cells and GD2^{-/-} SupT1. Target cell death was determined using flow cytometry, and FIGS. 8B and 8C show representative data from three replicates. Vδ2⁺γδT cells transduced to express anti-GD2 IgG1 had enhanced cytotoxicity against SupT1-GD2 but not against SupT1-wt (FIG. 8B). Supernatant from Vδ2⁺γδT cells transduced to express anti-GD2 IgG1 enhanced the cytotoxicity of non-transduced Vδ2⁺γδT cells against SupT1-GD2 but not against SupT1-wt (FIG. 8C).

Materials and Methods

Cell Lines

CAPAN-1, HELA and SupT1 cell lines were obtained from ATCC. SupT1-GD2 were generated by transducing wild type SupT1 with vector encoding GD2/GD3 synthase and isolating clones of successfully transduced cells.

Donor Selection for PBMC

PBMC were isolated from the blood of healthy donors aged 20-36y. Donors were screened for cross-reactivity between SFP constructs and expanding Vδ2 cells prior to inclusion. Donors where there was evidence of cross-reactivity were not used for cytotoxicity experiments.

Isolation and Pre-Stimulation Handling of Fresh PBMC

20 ml of whole blood were diluted with 10 ml PBS+500 μl 100 mM EDTA and layered on 20 ml Percoll. Interface PBMCs (20 min, 300xg, RT) were washed in PBS and re-suspended in 25 ml T-cell medium (X-VIVO 15 (Lonza BioWhittaker, Md., USA) supplemented with penicillin/streptomycin (100 IU/ml penicillin, 100 μg/ml streptomycin (Sigma-Aldrich, Mo., USA)) and cultured overnight before use.

Vδ2⁺ T-Cell Expansion

For specific Vδ2⁺ γδ T-cell expansion, PBMC were isolated as described above. They were cultured in RPMI-1640 medium supplemented with L-glutamine (2 mM, Sigma-Aldrich), penicillin/streptomycin (100 IU/ml penicillin, 100 μg/ml streptomycin (Sigma-Aldrich)) and 10% FCS (v/v, (Gibco, Mass., USA)). Vδ2⁺γδT cell expansion was stimulated using 5 μM zoledronic acid (Actavis, N.J., USA) and 100 IU/ml IL-2 (Aldesleukin, Novartis, Frimley, UK), which was added to PBMC suspension after PBMC isolation (day 1). IL-2 was replenished every 2-3 days by removing half of the media from the well and replacing with fresh media containing 200 IU/ml IL-2.

Construction of Retroviral Constructs

The gammaretroviral vector used in all constructs was SFG (Riviere et al 1995), pseudotyped with an RD 114 envelope. DNA fragments were amplified using the Phusion HT II polymerase according to the manufacturer's instructions (Thermo Scientific, Mass., USA). PCR was carried out in a PTC-200 DNA Engine (MJ Research, Mass., USA). PCR products were extracted from 1% agarose gels using the Wizard SV Gel & PCR Clean-Up kit (Promega, Wis., USA). Sample concentrations were determined using a NanoDrop ND-1000 spectrophotometer (Thermo Scientific, Mass., USA). The construct comprised one of a range of scFvs against a panel of targets including human GD2 (clone 14G2A) or human CEA (clone SM3EL) linked to the Fc portion of human IgG1.

In addition to the scFv-Fc fusion protein construct, RQR8 which is a marker bearing a CD34 epitope (Philip et al, 2014) was included, separated from the scFv-Fc fusion protein by a cleavable 2A peptide. This allows scFv-Fc fusion protein expressing cells to be detected by flow cytometry by staining using the anti-CD34 antibody clone QBend10.

Construction of Lentiviral Constructs

The lentiviral vector used for whole antibody transduction was pCCL (Dull et al 1998), pseudotyped with an RDPro envelope (Cosset et al 1995). DNA fragments were amplified using the Phusion HT II polymerase according to the manufacturer's instructions (Thermo Scientific, Mass., USA). PCR was carried out in a PTC-200 DNA Engine (MJ Research, Mass., USA). PCR products were extracted from 1% agarose gels using the Wizard SV Gel & PCR Clean-Up kit (Promega, Wis., USA). Sample concentrations were determined using a NanoDrop ND-1000 spectrophotometer (Thermo Scientific, Mass., USA). The construct comprised an anti-GD2 IgG1 (clone 14G2A).

In addition to the IgG1 construct, eGFP was included, separated from the IgG1 protein by a cleavable 2A peptide. This allows IgG1 expressing cells to be detected by flow cytometry.

Production of Viral Particles by Transfection 1.5×10⁶ 293T cells/100 mm² dish (Nucleon Delta Surface, Thermo Fisher) were plated at day 1 in 293T medium (D-MEM, 10% FCS (v/v)). γ-retroviral or lentiviral particles were produced by co-transfection of 293T cells at day 2 using Gene Juice Transfection Reagent (Novagen/Millipore, Mass., USA) in accordance with manufacturer's directions. Viral particle-containing supernatants were harvested at day 4; medium was replenished, and harvested at day 5. γ-retroviral supernatants were pooled, filtered (0.45-μm filter, Millipore) and directly used for transductions or stored overnight at 4° C. before use. Lentiviral supernatants were concentrated by ultracentrifugation and frozen for later use.

γ-Retroviral Transduction of T Cells

Transduction of T cells was carried out in Retronectin (Takara Bio, Tokyo, Japan) coated 24-well plates, which were pre-loaded with viral supernatant. 0.5×10⁶ T cells suspended in 0.5 ml T-cell medium+400 IU IL-2 were combined with 1.5 ml viral supernatant and centrifuged for 40 min, 1000xg at RT. Typically, 12×10⁶ T cells per donor were plated for transduction.

γδT-cell expansion was stimulated with 5 μM zoledronic acid (Actavis, N.J., USA) and 100 IU/ml IL-2 (Aldesleukin,) and transduction performed at day 5. At day 8 of culture (day 3 after transduction) cells were pooled, washed and plated at 2×10⁶ cells/ml in T-cell medium+100 IU IL-2/ml (24-well plates, Nucleon Delta Surface, Thermo Scientific, Mass., USA). Transduction efficiency was determined by flow cytometry at day 10 (day 5 after transduction).

Lentiviral Transduction of T-Cells

Transduction of T cells was carried out in 96-well plates, each well containing 0.3×10⁶ T cells suspended in 0.3 ml T-cell medium. Concentrated lentivirus was added and the plates centrifuged for 40 min, 1000xg at RT.

γδT-cell expansion was stimulated with 5 μM zoledronic acid (Actavis, N.J., USA) and 100 IU/ml IL-2 (Aldesleukin,) and transduction performed at day 2. At day 5 of culture (day 3 after transduction) cells were pooled, washed and plated at 1×10⁶ cells/cm² in T-cell medium+100 IU IL-2/ml. Transduction efficiency was determined by flow cytometry at day 7 (day 5 after transduction).

Cytotoxicity Assays

Target cell lines were labelled with CellTrace Violet (Thermo Fisher) before co-culture with effector cells at a 1:1 effector:target ratio. The media was either RPMI 1640+10% FCS+1% Pen/Step+1% L-glutamine supplemented with 100u/ml IL-2 or supernatant from scFv-Fc fusion protein secreting Vδ2 which had been cultured in the same media.

For assays involving effector cells sequestered behind semi-permeable membranes, Trans-Wells with 0.4 μm pore size (Thermo Fisher) used to separate the cells. An equal number of effectors were placed in the trans-well and the main well. Target cells were placed in the main well, with an effector:target ratio of 1:1 calculated on the cells in the main well.

After 18 h co-culture, cells were harvested and analysed by flow cytometry. Cell death was identified by staining using Ghost Red fixable viability dye (Tonbo Biosciences, San Diego, Calif.) on violet labelled cells.

References for Materials and Methods
I. Riviere, K. Brose, R. C. Mulligan, Effects of retroviral vector design on expression of human adenosine deaminase in murine bone marrow transplant recipients engrafted with genetically modified cells, Proc. Natd. Acad. Sci. U.S.A. 92, 6733-6737 (1995).
B. Philip, E. Kokalaki, L. Mekkaoui, S. Thomas, K. Straathof, B. Flutter, V. Marin, T. Marafioti, R. Chakraverty, D. Linch, S. A. Quezada, K. S. Peggs, M. Pule, A highly compact epitope-based marker/suicide gene for easier and safer T-cell therapy, Blood 124, 1277-1287 (2014).
T. Dull, R. Zufferey. M. Kelly, R. J. Mandel, M. Nguyen, D. Trono, L. Naldini. A Third generation lentivirus vector with a conditional packaging system. Journal of Virology, Nov 1998; 72(11):8463-71
F. L. Cosset, Y. Takeuchi, J. L. Battini, R. A. Weiss, M. K. Collins. High-titer packaging cells producing recombinant retroviruses resistant to human serum. Journal of Virology, Dec 1995; 69(12):7430-6

1. An immune cell that is capable of antibody-dependent cellular cytotoxicity (ADCC) and which comprises a nucleic acid sequence encoding an antigen binding molecule that comprises an antigen binding region.

2. The immune cell of claim 1, wherein the antigen binding region comprises a scFv, a Fab, a modified Fab, a Fab′, a modified Fab′, a F(ab′)2, a Fv, a dAb, a Fd, a dsFv, a ds-scFv, a scFv2, a bi-specific T-cell engager, a nanobody, a DARPin, an antibody mimetic, a diabody, a triabody, a tetrabody, or a polypeptide ligand for a receptor expressed on the surface of a cell that is targeted by the immune cell.

3. The immune cell of claim 1, wherein the antigen binding molecule is capable of binding to a Fc receptor.

4. The immune cell of claim 1, wherein the antigen binding molecule comprises a Fc region or a modified Fc region.

5. The immune cell claim 1, wherein the antigen binding molecule is an antibody, a scFv-Fc, a dAb-Fc, a heavy chain antibody, an IgNAR or a camelid antibody.

6. The immune cell of claim 1, wherein the immune cell is not an alpha beta T cell.

7. The immune cell of claim 1, wherein the immune cell is a gamma delta T cell or a NK cell.

8. The immune cell of claim 7, wherein the gamma delta T cell is a Vδ1+ gamma delta T cell, a Vδ2+ gamma delta T cell, or a Vδ1−/Vδ2- gamma delta T cell.

9. (canceled)

10. The immune cell of claim 1, wherein the immune cell is a myeloid cell, optionally wherein the myeloid cell is a macrophage, a basophil, an eosinophil, or a neutrophil.

11. (canceled)

12. The immune cell of claim 1, wherein the immune cell does not express a chimeric antigen receptor (CAR).

13. The immune cell of claim 1, wherein the antigen binding region is:
(a) capable of binding to an antigen that is expressed in the tumour microenvironment; and/or
(b) capable of binding to a tumour antigen, an endothelial antigen, or an immune cell antigen.

14. (canceled)

15. The immune cell of claim 1, wherein the antigen binding region is capable of binding to an antigen selected from a group consisting of CEA, B7-H3, TSHR, CD3, CD16, CD32, CD64, CD19, CD123, CD22, CD20, CD30, CD171, CS-1, CLL-1, CD33, EGFRvIII, GD2, GD3, BCMA, Tn Ag, PSMA, ROR1, FLT3, FAP, TAG72, CD38, CD44v6, EPCAM, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp1OO, bcr-abl, tyrosinase, EphA2, Fucosyl GMI, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OYTES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, LINGO1, CD70, IL13Ra2, MUC-16, PSCA, ROR1, and IGLL1:optionally wherein the antigen is CEA, B7-H3, CD20 or GD2.

16. (canceled)

17. The immune cell of claim 1, wherein the antigen binding molecule is an opsonin.

18. The immune cell of claim 1, wherein the immune cell expresses the antigen binding molecule.

19. The immune cell of claim 1, wherein the antigen binding molecule comprises a V_{H} domain encoded by SEQ ID NO: 1 and/or a V_{L} domain encoded by SEQ ID NO: 2, optionally wherein the antigen binding molecule is encoded by SEQ ID NO: 3.

20. (canceled)

21. The immune cell of claim 1, wherein the antigen binding molecule comprises a V_{H} domain encoded by SEQ ID NO: 4 and/or a V_{L} domain encoded by SEQ ID NO: 5, optionally wherein the antigen binding molecule is encoded by SEQ ID NO: 6.

22. (canceled)

23. The immune cell of claim 1, wherein the antigen binding molecule comprises a heavy chain encoded by SEQ ID NO: 20 and/or a light chain encoded by SEQ ID NO: 18, optionally wherein the antigen binding molecule is encoded by SEQ ID NO: 17.

24. (canceled)

25. The immune cell of claim 1, wherein:
(a) the nucleic acid sequence encodes two or more different antigen binding molecules; and/or
(b) the immune cell comprises two or more nucleic acid sequences each encoding a different antigen binding molecule.

26. (canceled)

27. A method of producing an immune cell according to claim 1, comprising introducing a nucleic acid sequence encoding an antigen binding molecule into an immune cell, optionally wherein the nucleic acid sequence is comprised in a vector, further optionally wherein the vector is a viral vector.

28. (canceled)

29. A method of treating disease in an individual, the method comprising administering to the individual a therapeutically effective number of immune cells according to claim 1, optionally wherein the disease is cancer, further optionally wherein the cancer is a solid tumour.

30. (canceled)

31. (canceled)

32. (canceled)
